(19) United States
(12) Patent Application Publication
(10) Pub. No.: US 2015/0079014 A1
(43) Pub. Date: Mar. 19, 2015

(54) Silicone-Based Prosthetic and Orthotic Liners with Antiperspirant and Methods of Forming the Same

(71) Applicant: OSSUR HF, Reykjavik (IS)

(72) Inventors: Thorvaldur INGVARSSON, Akureyri (IS); Sigurbjorg Saeunn GUDMUNDSDOTTIR, Reykjavik (IS); Bergthora SNORRADOTTIR, Reykjavik (IS)

(21) Appl. No.: 14/483,364

(22) Filed: Sep. 11, 2014

Related U.S. Application Data
(60) Provisional application No. 61/878,806, filed on Sep. 17, 2013.

(51) Int. Cl. A61F 2/78; A61L 27/54; A61L 27/34; A61L 27/30

(52) U.S. Cl. CPC A61F 2/7812; A61L 27/306; A61L 27/54; A61L 27/34; A61F 2002/5001
USPC 424/66; 424/65; 424/68

(57) ABSTRACT

Liners are adapted for use with a prosthetic or orthotic device, and provide a barrier and/or interface between a residual limb and a prosthetic socket, or between an orthotic device and the site of application. The prosthetic and orthotic liners include at least one antiperspirant, and methods of forming the same in an efficient, reliable manner. The liners may include aluminum-based antiperspirants such as Aluminum Zirconium Tetrachlorohydrex Glycine. The antiperspirants may be provided as a powder or anhydride, may first be constituted in a silicone-oil, and mixed under vacuum. The antiperspirant-oil mixture can be combined with silicone components and mixed under vacuum. The resulting complete mixture is then cast and cured into a silicone elastomer liner with antiperspirant dispersed and/or embedded.

### FIELD OF THE DISCLOSURE

This disclosure relates to liners adapted for use with a prosthetic or orthotic device, particularly those liners designed to provide a barrier and/or interface between a residual limb and a prosthetic socket, or between an orthotic device and the site of application. This disclosure relates to prosthetic and orthotic liners that include an antiperspirant feature.

### BACKGROUND

Using prosthetic liners is known in the art as exemplified by U.S. Pat. No. 4,923,474 granted May 8, 1990; U.S. Pat. No. 5,376,129 granted Dec. 27, 1994; U.S. Pat. No. 5,507,834 granted Apr. 16, 1996; U.S. Pat. No. 6,136,039 granted Oct. 24, 2000; U.S. Pat. No. 6,485,776 granted Nov. 26, 2002; U.S. Pat. No. 7,001,563 granted Feb. 21, 2006; U.S. Pat. No. 7,118,602 granted Oct. 10, 2006; U.S. Pat. No. 7,169,189 granted Jan. 30, 2007; U.S. Pat. No. 7,909,884 Mar. 22, 2011; U.S. Pat. No. 8,034,120 granted Oct. 11, 2011; and U.S. Pat. No. 8,052,760 granted Nov. 8, 2011, the entirety of each of which is incorporated by reference.

Elastomeric liners have been adapted to provide a soft, flexible interface and/or barrier between a residual limb (or post-operative stump) of an amputee and the hard socket to which a prosthetic device is secured. Such liners conform closely with the residual limb, accommodate all surface contours and sub-surface bone elements of the residual limb, and provide a comfortable cushion between the residual limb and the hard socket of the prosthesis to be fitted over the residual limb.

Under prior art teachings, such liners, sometimes called suspension liners or liner sleeves, may also function to secure the residual limb within the prosthetic socket member once the liner-covered residual limb is inserted into the socket in a close-fitting relationship; isolating the respective distal end areas of the hard socket and liner-covered residual limb from the atmosphere. A typical application may involve “rolling” an elastomeric liner onto the residual limb for a secure, form fit. The elastomer constituting the liner elastically and/or frictionally engages and remains attached to the residual limb so the limb is retained within the hard socket member in a comfortable, non-irritating manner. The liner may be thickened to provide a cushioning effect between the residual limb and the hard socket, which is typically custom-made to closely fit the residual limb.

As the prosthesis applies to the liner-covered limb, the air inside the socket of the prosthesis is pushed or otherwise forced out of the socket. Devices are usually provided to enable expulsion of air between the liner and the socket, and to isolate the respective distal ends of the socket and liner-covered residual limb from the atmosphere once the liner-covered residual limb has been fully inserted within the socket.

In such applications, the suspension of the prosthesis occurs, at least in part, due to the suction of the liner against the socket (establishing the term “suction socket” used for such liners). Upon application of a pulling force on the liner relative to the socket, suction is created in the distal end of the socket retaining the liner within the socket. In certain applications, it may be desirable to more positively secure the liner-covered limb within the socket by creating a hypobaric (vacuum) pressure within the distal end of the hard socket (i.e., between the distal end of the hard socket and the distal end of the liner inserted into the socket, with a residual limb within the liner sleeve). Opening the distal end of the socket to atmosphere releases the vacuum or hypobaric pressure within the socket to enable simple withdrawal of a residual limb with a liner sleeve thereon from the socket.

A pump or other device may be provided and/or utilized to evacuate the air and/or atmosphere from the distal end of the socket; between the distal end of a liner-covered limb and the distal end of a socket. A valve or other appropriate device typically is used to open and close the distal end of the socket to surrounding atmosphere.

Additional and/or alternative methods and mechanisms for securing a liner-covered limb within a socket, including using so called “umbrellas,” threaded members and/or receivers, prosthetic securing pins, bolts, screws, latches, and other locking and/or securing elements, as described in the U.S. Pat. No. 5,376,129, are known in the art.

A sleeve may also be worn as a primary or secondary means of suspension, sealing the top of the socket and making it airtight. The sleeve is rolled on over the prosthesis, extending onto the residual limb and sealing off the top of the socket to prevent air from entering or exiting the socket. Some sleeves incorporate a valve into the sleeve to release air during ambulation, particularly when performing a sitting or standing movement. Because such sleeves are may be airtight, they can cause problems with perspiration and discomfort.

Recent trends in prosthetic liners have been focused on increasing the comfort of such liners, enhancing their ability to conform to irregularities on the residual limb, accommodating a wider variety of residual limbs with fewer sizes of liners, and providing the amputee with a total feeling of comfort at the residual limb interface with the prosthesis, all while maintaining strength and durability of the liner. Silicone, rubber, gel, or other elastomer materials having suitable hardness (or softness), elongation, tensile, and other properties (e.g., sterilizability, porous, non-porous, easily cleanable, etc.) have been formulated and used successfully for liners. Prosthesis liners formed from silicone elastomeric materials have become widely used and are now well known in the art.

Silicone elastomer liners are typically air impermeable and may include a reinforcement layer intermediate the inner and outer surfaces of the liner sleeve body portion or externally to provide resistance against axial elongation of the elastomer constituting the liner sleeve body. While such features rarely restrict radial distension or stretching of the liner sleeve body, they often increase perspiration from sweat glands adjacent to the site of attachment.

While perspiration serves an important function in regulating the temperature of the body, many consider sweating to be a universal inconvenience. Prosthetic users however, may experience the detrimental effects of sweating to a degree larger than the average person. For instance, an amputee may expend more energy than the average person in carrying out the same activities; leading to increased perspiration. Walking with an artificial leg, for instance, requires more effort than walking on natural legs, which uses more energy, creating more body heat, to which the body responds by releasing sweat.

Another reason the amputee perspires more than the average person is due to losing skin surface on the body, through which sweat glands can release sweat to cool the body. An increase in perspiration from remaining glands must achieve the same cooling effect as those who retain their limb and/or all their skin surface area.

The prosthetic and prosthetic-related devices enhance perspiration. Tight-fitting, air-impermeable, silicone liners, for instance, preclude skin surface access to the cooling, sweat-evaporating effects of the surrounding air and provide an insulating barrier for retaining body heat and sweat. The socket of the prosthetic covers the residual limb with an air-tight, often bulky and even insulated piece of hardware that is suction-attached to the body. The prosthetic sleeve that covers the exterior of the socket member and extends onto the residual limb, seals off the top of the socket, making it airtight. The residual limb is isolated and enclosed in the prosthetic device, where air cannot reach it to evaporate perspiration from its skin's surface and cool the body.

Of primary concern for amputees is the perspiration or sweat that pools against the residual limb within the socket of the prosthetic limb. This buildup of sweat leads to skin irritations, blistering, bacteria build up, ingrown hairs, and several other undesirable skin and prosthetic fitting related issues. Such issues may require suspending the use of the prosthetic, causing personal, social, and even financial disruption and inconveniences.

Solutions to this problem are impractical or inconvenient, and are often short-lived and/or involve foregoing one desirable feature to obtain another. For instance, an amputee need only to remove the prosthetic and air out and/or dry off the residual limb, liner, socket, and sleeve any time perspiration accumulates. The repetitive removal and care of the limb and device required to avoid perspiration-related issues or problems, however, may negate many of the conveniences that the device provided.

Some amputees apply a prescription or over-the-counter antiperspirant to help reduce perspiration. These antiperspirants generally are thought to reduce perspiration by associating with water molecules from the surrounding environment, entering the sweat gland duct cells with which it comes in contact, and causing it to swell, eventually pinching off the sweat gland duct, limiting the perspiration that can be secreted from a sweat gland. While generally effective, these topically antiperspirant agents provide only temporary assistance in controlling perspiration. To retain their effectiveness, antiperspirants must be reapplied as the active ingredient is depleted from the powder, lotion, gel, aerosol spray, or other carrier. Reapplication of antiperspirant to the residual limb requires the same impractical and inconvenient device removal process addressed above.

Further attempts to solve this problem have been directed at mechanical mechanisms for eliminating the moisture produced through openings in the liner and socket. Such alterations are designed to manage perspiration in prosthesis by replacing traditional components with air and sweat permeable liners, sockets, and sleeves made from porous or otherwise ventilated material that allows air and/or moisture in and out of the system for skin surface cooling and sweat evaporation. In prosthetics, see for example U.S. Pat. No. 8,382,852.

Other attempts include replacing the silicone liner with a moisture-wicking material that pulls or draws sweat away from the surface of the skin. Some prosthetic “socks” include permanent, antimicrobial, moisture-wicking silver fibers woven into the fabric of the sock.

There is a significant need for a practical, convenient solution for controlling perspiration with prosthetics that does not require the user to forego the benefits of elastomeric liners.

### SUMMARY

Various embodiments concerning liners adapted for use with a prosthetic or orthotic device, particularly those liners designed to provide a barrier and/or interface between a residual limb and a prosthetic socket, or between an orthotic device and site of application, are disclosed. Embodiments of the disclosure overcome or solve one or more of the foregoing or other problems in the art with prosthetic and/or orthotic liners that include at least one antiperspirant, and methods of forming the same in an efficient, reliable manner.

For instance, one or more embodiments include a liner for use with a prosthetic or orthotic device, wherein the liner includes an antiperspirant dispersed in carrier material. The carrier material may be configured for attachment to a body part and the carrier material and antiperspirant may be configured to permit the antiperspirant to leach from the carrier and onto the body part to which the carrier material is attached. The antiperspirant may be capable of reducing perspiration of the body part to which the carrier material is attached by at least 20% compared to a carrier material that includes no antiperspirant dispersed. In at least one embodiment, the antiperspirant includes a hygroscopic and/or hydrophilic substance, an aluminum-based compound, an aluminum salt, and/or aluminum zirconium tetrachlorohydrex glycine. In certain embodiments, the carrier is capable of elastic attachment to the body part, the carrier material comprises a silicone-based compound, and/or the carrier comprises a cured two-component silicone-based compound.

One or more embodiments include a method of manufacturing a liner for use with a prosthetic or orthotic device. The method includes incorporating and/or embedding an antiperspirant into or within a polymeric and/or elastomeric material and forming the polymeric and/or elastomeric material into a liner, wherein the liner is configured for use with a prosthetic or orthotic device. The method may further include combining an antiperspirant agent with a silicone oil to form a first mixture and mixing the first mixture under vacuum to form a balanced mixture. Air may be substantially removed from the first mixture during mixing, and the balanced mixture may include a combination of the antiperspirant agent and the silicone oil, from which air has been substantially removed. The method may also include adding at least one silicone component to the balanced mixture to form a second mixture and mixing the second mixture under vacuum to form a complete mixture. Air may be substantially removed from the second mixture during mixing, and the complete mixture may include a combination of the balanced mixture and the at least one silicone component, from which air has been substantially removed.

The method may also include curing the complete mixture to form a silicone product such that the antiperspirant agent is dispersed in the silicone product. In at least one embodiment, the antiperspirant agent comprises an aluminum salt and/or aluminum zirconium tetrachlorohydrex glycine. In some embodiments, the silicone product comprises a liner for use with a prosthetic or orthotic device, and/or the liner can reduce perspiration of a body part to which the liner is attached by at least 20% compared to a liner that includes no antiperspirant agent dispersed.

Additional features and advantages of illustrative and/or exemplary embodiments will be in the description which follows, and in part will be obvious from the description, or may be learned by the practice of such exemplary embodiments. The features and advantages of such embodiments may be realized and obtained with the instruments and combinations pointed out in the appended claims. These and other features will become more fully apparent from the following description and appended claims, or may be learned by the practice of such illustrative and/or exemplary embodiments as set forth.

### BRIEF DESCRIPTION OF THE DRAWINGS

To describe the manner in which the above-recited and other advantages and features of the disclosure can be obtained, a more particular description briefly described above will be rendered by reference to embodiments which are illustrated in the appended drawings. Understanding that these drawings depict only typical embodiments and are not therefore to be limiting of its scope, the embodiments will be described and explained with additional specificity and detail through the accompanying drawings in which:

FIG. 1 illustrates an embodiment of a liner for use with a prosthetic device.

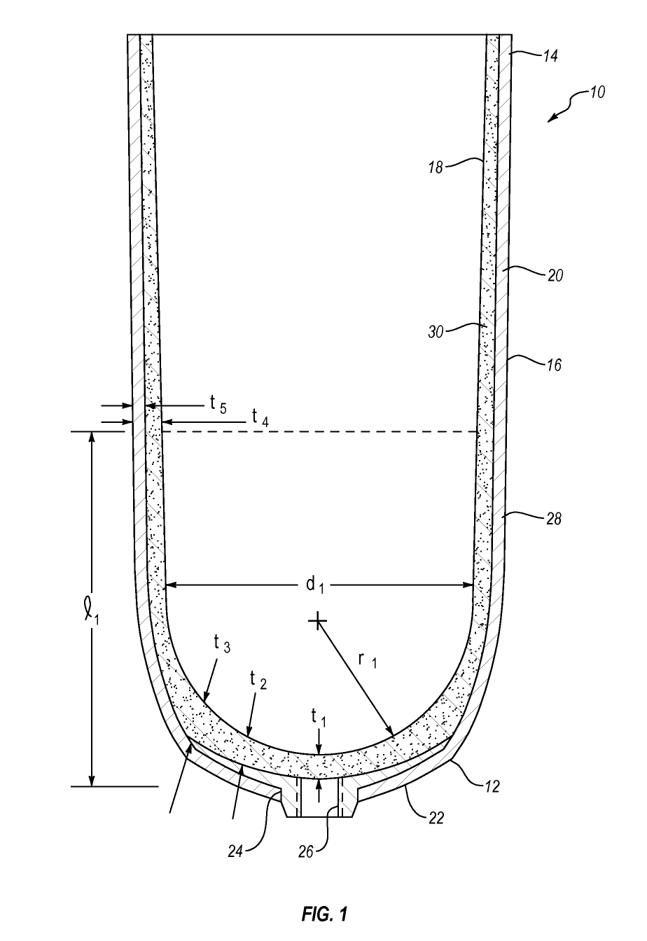

### DETAILED DESCRIPTION OF ILLUSTRATIVE EMBODIMENTS

Various embodiments are associated with liners adapted for use with a prosthetic or orthotic device, those liners designed to provide a barrier and/or interface between a residual limb and a prosthetic socket, or between an orthotic device and site of application. The embodiments may overcome or solve one or more of the foregoing or other problems in the art with prosthetic and/or orthotic liners that include at least one antiperspirant, and methods of forming the same in an efficient, reliable manner.

In at least one embodiment, an elastomeric liner that includes at least one antiperspirant or antiperspirant agent is provided. The liner may include any natural, synthetic, polymeric and/or elastomeric material known in the art. In certain embodiments, the liner includes a silicone elastomeric substance and/or other material.

As used herein, “silicone” and similar terms include any of a group of semi-inorganic polymers based on the structural unit R2SiO, where R is an organic group, and which is characterized by wide-range thermal stability, high lubricity, extreme water repellence, and/or physiological inertness and which may be used in adhesives, lubricants, protective coatings, paints, electrical insulation, synthetic rubber, and prosthetic replacements for body parts.

As used, “silicone oil” and the like include any liquid polymerized siloxane (formed with a backbone of alternating silicon-oxygen atoms ( . . . Si—O—Si—O—Si . . . )) and which may include one or more organic side chains attached to the tetravalent silicon atoms, but not to the divalent oxygen atoms fully committed to forming the siloxane chain. In some cases, silicone oils are non-flammable and exhibit high temperature-stability and good heat-transfer characteristics. Some silicone oils, such as simethicone, illustratively, are potent anti-foaming agents due to their low surface tension.

As used, the terms “antiperspirant,” “antiperspirant agent,” “antiperspirant compound,” and/or the like include any substance capable of preventing or reducing perspiration through any mechanism, known or unknown.

In general, and as understood more fully, a residual limb or post-operative stump of an amputee is presented illustrative as one use or basis for certain embodiments. It is to be understood, however, that other applications, forums, and/or uses also exist for various embodiments. Illustrative examples include congenital, genetic, and/or other bodily defects or deformities which facilitate, accommodate, and/or necessitate prosthesis (or using a prosthetic device) and/or orthosis (or using an orthotic device), or any other application in which an external device is attached to a portion or part of the body, particularly where eccrine or apocrine sweat glands are located on the body.

One or more embodiments include a liner for use with a prosthetic or orthotic device, wherein the liner includes an antiperspirant dispersed in a carrier material (i.e., a carrier). In at least one embodiment, the carrier material is configured for attachment to a body part. The body part may be a residual limb or post-operative stump of an amputee. One will appreciate, however, that the present disclosure is not so limited. A body part according to the present disclosure is given broad interpretation; as in any portion, part, or component of any member, element, unit, or matter. For instance, a liner and/or carrier material according to some embodiments may be configured for attachment to a portion of a display member or figure, such as a manikin; or to a non-human animal, such as a dog, cat, horse, and/or other living organism, without departing from the scope of the disclosure.

In at least one embodiment, the carrier material and the antiperspirant are configured to permit the antiperspirant to leach from the carrier material and onto the body part to which the carrier material is attached. In certain embodiments, the carrier and antiperspirant are configured to permit the antiperspirant to leach from the carrier and onto the body part to which the carrier is attached over an extended period. An extended period of time according to some embodiments in this disclosure may include:

(1) any period of time, whether measured in hours, minutes, or some other unit of time, that is longer and/or greater than (a) a typical and/or average work day in the United States, (b) the clinically-determined and/or advertised period of effectiveness and/or perspiration control for a commercially available product containing the same or similar active ingredient(s) as the antiperspirant, (c) the clinically-determined and/or advertised period of effectiveness and/or perspiration control for a comparable product containing the same or similar active ingredient(s) as the antiperspirant;

(2) a period of time longer and/or greater than (a) one day, (b) two days, (c) three days, (d) one week, (e) two weeks, (f) one month, (g) six months, (h) one year, (i) 6 hours, and/or (j) 12 hours; and/or

(3) any period of time, depending on the activity being performed, that is longer and/or greater than the determined average period of effectiveness for the antiperspirant, for a test subject performing the same activity, when the antiperspirant is applied directly to the body part, whether the body part is subsequently covered with the carrier or not.

One will appreciate, however, that the present disclosure is not so limited and that an extended period of effectiveness may be activity- and/or user-specific, and may include any period of time longer that an anticipated period of effectiveness for the antiperspirant without the carrier.

In some embodiments, the carrier and antiperspirant are configured to permit the antiperspirant to leach from the carrier and onto the body part to which the carrier is attached over a period of time determined by the rate, amount, and/or quantity of perspiration and/or other form of moisture to which the carrier (and/or antiperspirant) come in contact. Leaching rates and/or rates of diffusion of the antiperspirant from the carrier may also or alternatively be determined by porosity of the carrier, solubility of the antiperspirant, concentration of the antiperspirant contacting at least one exterior surface of the carrier, and other factors that contribute to the rate at which molecules, compounds, or other substances move, transfer, and/or diffuse through a material.

In certain embodiments, the carrier and antiperspirant are configured to permit the antiperspirant to leach from the carrier and onto the body part to which the carrier is attached over time determined by the rate of uptake, diffusion, and/or movement of the antiperspirant into the body part to which the carrier is attached. For instance, the leaching rates and/or rates of diffusion of the antiperspirant from the carrier may depend on the speed or rate at which a person sweats, at which the antiperspirant is drawn into the sweat gland duct cells adjacent to the surface of the carrier, or both.

In some embodiments, the antiperspirant and/or antiperspirant-embedded carrier can reduce perspiration (and/or sweating) of the body part to which the carrier material is attached by at least 20% compared to a control substance and/or a carrier material that includes no antiperspirant dispersed. One will appreciate, however, that the present disclosure is not so limited, and that less effective antiperspirants (and/or antiperspirant-embedded carriers) are also contemplated. In at least one embodiment, the antiperspirant and/or antiperspirant-embedded carrier material can reduce perspiration of the body part to which the carrier material is attached by at least 5%, at least 10%, at least 30%, and/or at least 50% compared to a control substance and/or a carrier material that includes no antiperspirant dispersed.

In at least one embodiment, the antiperspirant and/or the carrier material in which the antiperspirant is dispersed (i.e., the antiperspirant-embedded carrier/carrier material) can reduce perspiration of the body part to which the carrier material is attached by at least 20% in at least 50% of tested users, compared to a control substance and/or a carrier material that includes no antiperspirant dispersed. In certain illustrative embodiments, the antiperspirant and/or antiperspirant-embedded carrier material can reduce perspiration of the body part to which the carrier material is attached by an average of about 57% compared to a control substance and/or a carrier material that includes no antiperspirant dispersed.

In one or more embodiments, the antiperspirant may include a hygroscopic substance (i.e., a material, solution, or other substance that readily binds, uptakes, absorbs, attracts, and/or associates with moisture and/or water molecule(s) from the atmosphere) and/or hydrophilic substance.

In at least one embodiment, the antiperspirant includes an aluminum based compound, particularly an aluminum salt. In certain embodiments, the antiperspirant and/or aluminum salt includes aluminum zirconium tetrachlorohydrex glycine (AZCH). One will appreciate, however, that other antiperspirants, aluminum based compounds, agents, materials, substances, and/or aluminum salts are contemplated.

Illustrative examples of antiperspirant and/or aluminum based compounds, agents, substances, and/or materials, and/or aluminum salts, include: Aluminum Chloride, Aluminum Chlorohydrate, Aluminum Chlorohydrex Polyethylene Glycol Complex, Aluminum Chlorohydrex Propylene Glycol Complex, Aluminum Dichlorohydrate, Aluminum Dichlorohydrex Polyethylene Glycol Complex, Aluminum Dichlorohydrex Propylene Glycol Complex, Aluminum Sesquichlorohydrate, Aluminum Sesquichlorohydrex Polyethlene Glycol Complex, Aluminum Sesquichlorohydrex Propylene Glycol Complex, Aluminum Zirconium Octachlorohydrate, Aluminum Zirconium Octachlorohydrex Glycine Complex, Aluminum Zirconium Pentachlorohydrate, Aluminum Zirconium Pentachlorohydrex Glycine Complex, Aluminum Zirconium Tetrachlorohydrate, Aluminum Zirconium Tetrachlorohydrex Glycine Complex, Aluminum Zirconium Trichlorhydrate, Aluminum Zirconium Trichlorohydrex Glycine Complex, Aluminum Zirconium Trichlorohydrex Glycine Complex, Aluminum Sulfate, Aluminum Sulfate Buffered, Aluminum Sulfate Buffered With Sodium Aluminum Lactate, and similar substances.

In one or more embodiments, the carrier is capable of elastic attachment to the body part. The carrier may be capable of elastically and/or frictionally engaging and remaining attached to the residual limb of an amputee. In at least one embodiment, the carrier material includes a silicone-based compound. For instance, the carrier material may include a silicone-based, elastomeric liner for a prosthesis, which includes a soft inner silicone elastomer layer and a relatively harder outer silicone elastomer layer, with both layers being formulated to provide desired physical characteristics of the liner. One will appreciate, however, that the disclosure is not limited to a two-layer silicone-based liner and/or elastic attachment. The liner and/or carrier may include a single or multiple layers, and may comprise any polymeric, elastomeric, and/or other suitable material.

Certain embodiments may include a material or substance into which the antiperspirant is initially dissolved, suspended, embedded, or otherwise dispersed. For instance, the antiperspirant may initially be suspended in a silicone oil carrier. The carrier may also or include a material into which the antiperspirant is ultimately dissolved, suspended, embedded, or otherwise dispersed. For instance, the antiperspirant may ultimately be embedded into an elastomeric liner.

An elasticity controlling matrix material and a prosthesis connecting element may be in the distal end area of the liner. The softer inner layer may closely conform to the body part or residual limb to be fitted to and/or within a prosthetic or prosthetic device while the harder outer layer may provide durability and strength for the liner.

### Illustrative Example 1

In the drawing, a silicone elastomer liner 10 intended for use between a residual limb and a prosthesis (not illustrated) includes a distal end 12, a proximal end 14 and an axially extending mid-portion 16 between the distal and proximal ends 12, 14. The liner may be air-tight when donned over a residual limb (not illustrated).

The entire liner 10 is formed of two layers of silicone elastomer to be described in more detail below, the inner layer 18 extending throughout the inner surface of the liner 10 and the outer layer 20 overlaying and contiguous with the outer surface of the inner layer 18, the interface between the layers 18 and 20 constituting a seamless, integral, permanent connection between the layers.

Preferably, at least the inner layer 18 tapers in thickness from a relatively thick cross-section at the distal end 12 of the liner to a thinner cross-section at the proximal end 14 of the liner. The outer layer 20 may have a uniform thickness along the mid-portion 16 with a thickened distal end portion 22, although its outer configuration can be varied by a suitable outer mold cavity.

Optionally, a relatively rigid prosthesis connecting “umbrella” element 24 having a concave curved configuration as shown and a threaded socket 26 for receiving a prosthesis locking pin (not illustrated) may be at the distal end 12 of the liner 10, preferably embedded in the silicone elastomer between the inner and outer layers 18, 20. The connecting element 24 may be intimately bonded to the silicone elastomer constituting the liner 10. Other connection and/or suspension elements, members, and/or mechanisms are known in the art and contemplated.

An elasticity controlling matrix material 28 is provided between the layers 18, 20 in the distal end area 12 of the liner 10, the matrix reinforcement material being relatively compliant in a radial direction and substantially rigid or inelastic in the axial direction. The matrix material 28 in a preferred embodiment may extend over the distal or outer side of the prosthesis connecting element 24 using an assembly and molding process to be described below.

In at least one embodiment, embedded within at least the inner layer 18, an antiperspirant 30 is dispersed. In some embodiments, embedded within at least the outer layer 20, an antiperspirant 30 is dispersed. In a preferred embodiment, antiperspirant 30 is dispersed evenly and/or uniformly throughout the inner layer 18 and optionally, the out layer 20.

The liner 10 is fabricated in a sufficient number of sizes to accommodate various sizes of residual limbs. In use, a liner of the type described may be rolled up from the proximal to the distal end, placed over the distal end of the residual stump and rolled back up or “donned” over the stump like a stocking. This procedure and the benefits achieved are described in the Klasson and Kristinsson U.S. Pat. No. 4,923,474.

One will appreciate, however, that the disclosure is not so limited and that a two-layered silicone elastomeric liner is illustrative only. For instance, a liner according to certain embodiments may comprise a single layer or a plurality of layers. In at least one embodiment, the liner includes a single layer of silicone elastomer with or without an outer layer and/or cover comprising a different silicone or non-silicone material or substance. A single layer silicone elastomer with an outer textile layer or cover is embodied and as more fully disclosed in U.S. Pat. No. 7,025,793, the entirety of which is incorporated by reference.

A liner adapted to provide an interface between a residual limb and a prosthetic socket, and having outer and inner surfaces is disclosed. The liner may include an elongated conical body portion formed from at least one material segment defining the liner inner and/or outer surface. The at least one material segment may be at least radially and/or longitudinally elastically extensible from a relaxed non-extended condition, and may include proximal and distal end areas. The liner may include a layer of polymeric material disposed on the at least one material segment and defining the liner inner and/or outer surface.

Optionally, the liner may include an outer layer comprising a textile or other material. The outer layer may prevent, reduce, or otherwise inhibit an antiperspirant dispersed and/or embedded within the liner from leaching from or through the outer layer.

Optionally, one or more resilient seal elements protruding radially from the liner outer surface may be included. The seal element(s) may extend around at least one peripheral portion of the liner body portion. A pair of opposed annular recesses may be adjacently above and/or below each of the one or more seal elements.

### Illustrative Example 2

In one or more embodiments, the silicone elastomer used to form the inner layer of the liner and/or carrier comprises a vinyl terminated polydimethylsiloxane (vinyldimethylsiloxy terminated dimethylpolysiloxane) cured or vulcanized by reaction with a suitable cross-linker. The silicone elastomer may be reinforced with silica (preferably fumed silica having a surface area of 200 m.sup.2/g.) and this may increase the strength of the cured or cross-linked silicone. The degree of crosslinking can be adjusted to some extent by adjusting the concentration of cross-linker. The component in the elastomer that the cross-linker reacts with may be a vinyl group on the ends of the polysiloxane. The end groups on the polysiloxane may also control the viscosity of the silicone and the concentration can be varied to allow a formulator to manufacture polysiloxanes of various viscosities.

Specifically, more than one end blocking moiety may provide control of viscosity and level of crosslinking somewhat independently of each other. A preferred viscosity may be in the range of 90,000-100,000 cPs. Non-functional endblocking (trimethyl siloxy) may also be used with vinyl endblocking (vinyldimethyl siloxy) to allow the production of a polysiloxane with a lower vinyl concentration than would otherwise be necessary if vinyl endblocking was used exclusively. This technique may permit the production of low viscosity silicones having a somewhat lower density crosslinking than is commonly used in silicone elastomers.

The organopolysiloxane may contain silyl groups of the formula R.sub.1 R.sub.2-SiO and end blockers of the formula R.sub.3 R.sub.4 R.sub.5-SiO.sub.05, R.sub.1 and R.sub.2 groups independently are lower alkyl of 1 to 6 carbons, phenyl or trifluoropropyl. Preferably the R.sub.1 and R.sub.2 groups may both be methyl. Therefore, in a preferred embodiment of the composition the R.sub.1 R.sub.2-SiO group represents dimethylsiloxane. R.sub.3, R.sub.4 and R.sub.5 groups independently are lower alkyl of 1 to 6 carbons, phenyl, vinyl, allyl or other olefinic group having up to 4 carbons. In a preferred embodiment, R.sub.4 and R.sub.5 are methyl and R.sub.3 is vinyl or methyl. Optionally, the molar concentration of vinyl in R.sub.3 may be varied from as high as 100% vinyl to as low as 30% vinyl, the remaining fraction being methyl. Preferentially, R.sub.3 is vinyl at 80% and methyl at 20% concentration.

The composition may also contain trimethyl silyl treated silica as a reinforcer or reinforcing element in the weight ratio of approximately 12 to 45 parts of reinforcer to 100 parts of polymer. A preferred embodiment contains 17 parts of silica to 100 parts of polymer. The silica may be treated with a reagent to neutralize the active sites on its surface, usually using hexamethyldisilazane.

Organopolysiloxane of lower viscosity, 100 to 10,000 cP may also be added to the composition to reduce viscosity, modulus, and tensile set. The quantity of organopolysiloxane can be adjusted to give the desired property profile. A preferred organopolysiloxane may be trimethyl siloxy terminated dimethylpolysiloxane with viscosity of 1000 cP.

The elastomer may illustratively be made from, of, and/or in (or may comprise) two components, called part “A” and “B”. Part A is constituted of (or may comprise) the polydimethylsiloxane that is vinyl and methyl terminated. A platinum catalyst may be used, the catalyst optionally comprising a complex of platinum with vinyl-containing oligosiloxanes (complex of platinum and divinyltetratramethyldisiloxane with typical levels of active platinum of 5 to 50 parts per million.

Part B of the two components of the elastomer may include a polydimethylsiloxane and silica identical to that in part A. This part B may also include a polydimethylsiloxane with hydrogen on the chain commonly called methyl hydrogen which acts as a crosslinker. With the mass of polydimethylsiloxane and silica constituting 100 parts, crosslinker concentration can vary from as low as 0.3 to as high as 4 parts per hundred parts. A crosslinking inhibitor may also be in part B that comprises an oligosiloxane with high concentration of vinyl-containing substituents of any of the class of compounds known as acetylinic alcohols. A preferred inhibitor may be tetravinyl tetramethyl cyclotetrasiloxane. The inhibitor may be used in concentrations as low as 0.02 parts per hundred parts to as high as 0.5 parts per hundred parts.

An example of a preferred silicone elastomer may be obtainable from NuSil Technology of Carpinteria, Calif., under product designation CF13-2188.

A preferred outer layer 20 of the liner 10 may be constituted of and/or include a vinyl terminated polydimethylsiloxane cured or vulcanized by reaction with a suitable crosslinker. The silicone may typically be reinforced with silica, the degree of crosslinking optionally being controlled by the concentration of crosslinker. This silicone material is also obtainable from NuSil Technology of Carpinteria, Calif., under product designation CF3-2188-1. The silicone elastomer may be addition-cured using a platinum catalyst of the type described above with the NuSil product CF13-2188 silicone elastomer. The silicone elastomer used in the outer layer also may be provided as two components, parts “A” and “B”.

Part A may be made from polydimethylsiloxane optionally vinyl terminated and a second polydimethylsiloxane optionally trimethyl terminated. Trimethyl terminated polymer may be at a concentration of 1-10% and silica may be in the formulation for reinforcement. The viscosity of the uncured part A elastomer used to form the outer layer of the liner is 250,000-800,000 cPs. The silica may be treated with a reagent to neutralize the active sites on its surface using, preferably, hexamethyldisilazane, with the concentration of silica being 12-45 parts per hundred parts of polysiloxane, with a preferred concentration being 25 parts per hundred per one hundred parts of polysiloxane. Titanium dioxide may be added to the part A component of the silicone elastomer in concentration sufficient to color it opaque white in a concentration of 2-15%, if a white color is desired. A platinum catalyst may be added to the part A component, the catalyst optionally comprising a complex of platinum and divinyltetramethyldisiloxane with a level of active platinum typically from 5-50 parts per million.

The part B component of the CF3-2188 silicone elastomer may comprise polydimethylsiloxane and silica identical to that used in part A described above and a crosslinker. Typically, part B contains no pigment, and is translucent. Assuming the mass of polydimethylsiloxane and silica to constitute 100 parts, polydimethylsiloxane with hydrogen on the chain (commonly called methyl hydrogen) is the crosslinker in concentrations extending from 0.3 to 4.0 parts per hundred. A crosslinking inhibitor may be added to part B in an oligosiloxane with a high concentration of vinyl-containing substituents or any of the class of compounds known as acetylinic alcohols to control rate of crosslinking A preferred inhibitor may be tetravinyl tetramethyl cyclotetrasiloxane in concentrations from about 0.02 parts per hundred to 0.5 parts per hundred.

Though the present disclosure is not limited to a two-layer liner, method, or system, when inner and outer layers are formed using the silicone elastomers described above, the following physical characteristics of the layers may be obtained:

Characteristic; Inner Layer; Outer Layer

Transparency; Translucent; Translucent

Shore A; Not Measurable; 6

Shore 00; 32-45; 52

Tensile Strength (min) (p/in); 233; 350

Tensile Strength (max) (p/in); 500; 551

Elongation (%); 1000; 1150

Modulus 100% (psi); 8; 21

Modulus 200%; 26; 53

Modulus 500%; 61; 139

Tear Strength (nick) (p/in); 49 (0.002″); 84 (0.005″)

The elasticity controlling matrix material 28 can be 12 cm and/or 15 cm lengths, and this material may be a white woven polyamide stockinette. The matrix material may be fully compliant and stretchable in the radial direction within the range of normal liner distension but is substantially inelastic or non-stretchable in the longitudinal direction when installed in the liner.

It will be seen from the shore hardness properties of the inner and outer layers 18, 20 that the inner layer may be considerably softer than the outer layer, and have a lower tear strength than the outer layer. The outer layer may possess greater hardness and tear strength, which reinforces the softer inner layer and provides a product that is both strong and durable, while very comfortable for the user. The ability of the inner layer 18 to conform to the skin surface of the residual limb of the user reduces any gaps between the skin and the inner layer which reduces perspiration between the residual limb and the inner surface of the liner, a characteristic highly desirable in a liner.

Friction properties of the inner, softer liner against the skin of the residual limb are such that a higher shear force is needed to cause slippage between the inner layer and the skin as compared with prior art silicone elastomer liners, which enhances the suspension properties and comfort of the liner.

The viscosity of the liquid silicone elastomer used to form both layers 18 and 20 is low enough to allow rapid injection molding of large parts. The preferred viscosities may be 90,000 to 100,000 cPs. The formulation of the outer layer 20 may be varied, provided that the physical property characteristics of the outer layer as described above remain essentially consistent, particularly regarding a Shore 00 hardness which must be higher than that of the inner layer 18. Typically, the tensile and tear strengths of the outer layer will be higher than these strengths of the inner layer. It may also be preferred that the outer layer have a higher elongation and modulus than the inner layer.

Coloration of the liner is optional, and one example has been described above wherein the inner layer is colored white and the outer layer is translucent.

Typical preferred dimensions of exemplary liners constructed under certain embodiments are shown below, referring to the drawing that indicates the locations where the measurements are taken.

Small; Large

t.sub.1 (mm): 6; 9

t.sub.2 (mm): 5; 4.5

t.sub.3 (mm) 6.5; -

t.sub.4 (mm) 2.7; 1.7

t.sub.5 (mm) 2.4; 2.2

r.sub.1 (cm) 40; 70

l.sub.1 (cm) 8-9; 13-14

l.sub.t (cm) 34; 40

d.sub.1 (cm) 9; 28.6

W (gm) 230; 880

where the measurements are described:

t.sub.1—inner layer thickness

t.sub.2—inner layer thickness

t.sub.3—inner layer thickness

t.sub.4—inner layer thickness

t.sub.5—outer layer thickness

r.sub.1—radius of liner end

l.sub.1—length of matrix

l.sub.t—total length of liner

d.sub.1—inside diameter

W—weight of liner

In at least one embodiment, the carrier material includes a cured two-component (or two-part) silicone-based compound. One will appreciate, however, that the present disclosure is not so limited, and that single and multi-component carrier material(s) and/or compound(s) are contemplated. Embodiments may be formed from single and or multi-component silicone and/or other elastomer(s), or other polymeric, elastomeric, and/or non-silicone material(s). In certain embodiments, the liner and/or carrier may include a single layer of said material(s), with or without at least one textile or other cover. In one or more embodiment, the cover may serve, function, or otherwise be configured as an outer barrier of the liner or carrier material. Such a barrier may prevent, reduce, or otherwise inhibit an antiperspirant dispersed and/or embedded within the liner and/or carrier material(s) from leaching from, out, or through the cover or barrier. Additional covers and/or barriers are also contemplated.

### Illustrative Example 3

In at least one embodiment, the antiperspirant-embedded carrier comprises a silicone liner for use with prosthetic or orthotic devices that contain aluminum zirconium tetrachlorohydrex gly (AZCH), which functions as an antiperspirant, embedded. A silicone liner that can be used with both prosthetic and orthotic devices having AZCH embedded in the silicone is disclosed. The silicone and AZCH formulation functions to reduce the perspiration generated on the skin and or other surface of the body part adjacent to the liner. The mechanism is believed to involve a slow leaching of the AZCH out of the silicone then taken up by the skin. AZCH is a hydrophilic and/or hygroscopic material (because it readily associates with and uptakes moisture from the atmosphere or surroundings) used in commercial antiperspirants thought to bind to water, expand and subsequently block the release of sweat from the pores in the skin.

Clinical trials with silicone liners containing AZCH compared with similar liners not containing AZCH show the combination is effective in reducing perspiration of the skin by up to 20-30% in 50% of the tested users. Reduction of the perspiration generated by the skin adjacent to a liner surface directly affects the comfort and fit of the liner and reduces the skin irritation experienced by the user. Such a system of perspiration control can be used where perspiration causes problems; particularly with liners for sockets that secure prosthetic limbs or liners or surfaces that contact the skin or surface on orthotic devices.

Method Description

One or more embodiments include a method of manufacturing a liner for use with a prosthetic or orthotic device. The prosthetic and/or orthotic liner may include at least one antiperspirant (or antiperspirant agent, compound, substance, and/or material). The antiperspirant may be dispersed and/or embedded within the liner.

The method may include providing an antiperspirant and/or antiperspirant agent, combining the antiperspirant agent with a silicone oil, liquid, or other carrier to form a first mixture, and mixing the first mixture under vacuum or reduced pressure to form a balanced mixture. Air may be substantially removed from the first mixture during mixing, and the balanced mixture may include a combination of the antiperspirant agent and the silicone oil, from which air has been substantially removed. One will appreciate, however, that the disclosure is not so limited, and that in certain embodiments, the first mixture need not be mixed under vacuum or reduced pressure.

At least one embodiment may also include adding at least one silicone component to the balanced mixture to form a second mixture and mixing the second mixture under vacuum or reduced pressure to form a complete mixture. Air may be substantially removed from the second mixture during mixing, and the complete mixture may include a combination of the balanced mixture and the at least one silicone component, from which air has been substantially removed. One will appreciate, however, that the disclosure is not so limited, and that in certain embodiments, the second mixture need not be mixed under vacuum or reduced pressure.

One will appreciate, however, that in at least one embodiment, the antiperspirant is not combined with silicone oil, liquid, or other carrier to form a first mixture. Such an embodiment may include combining the antiperspirant directly with the at least one silicone component to form a mixture and mixing the mixture under vacuum or reduced pressure to form a complete mixture. In certain embodiments, however, the mixture need not be mixed under vacuum or reduced pressure.

Some embodiments may also include casting and/or curing the complete mixture to form a silicone or other product such that the antiperspirant agent is dispersed and/or embedded in the silicone or other product. The antiperspirant agent may comprise an aluminum based compound, an aluminum salt, and/or aluminum zirconium tetrachlorohydrex glycine (AZCH). The antiperspirant agent may be provided as a powder or anhydride. In some embodiments, the antiperspirant agent, when provided as a powder or anhydride, may first be constituted in a silicone-oil, liquid, or other carrier and/or mixed under vacuum.

In at least one embodiment, the silicone product may comprise a liner for use with a prosthetic or orthotic device. In certain embodiments, the antiperspirant and/or liner can reduce perspiration of a body part to which the liner is attached by at least 20% compared to a liner that includes no antiperspirant agent dispersed.

It will be appreciated that silicone and silicone-oil are illustrative only, and that the present disclosure is not so limited. Other formulations, including polymeric substances of other elemental bases are contemplated.

### Illustrative Example 4

In at least one embodiment, first, the AZCH is mixed with a silicone-oil and then a vacuum is applied on it. Next, the AZCH-oil mixture is combined with the silicone components (A and B) and the vacuum is applied on it again. The vacuum applies to release any air that often mixes when the stirring of the oil and AZCH is conducted. The vacuum does not appear to cause solvent evaporation. The vacuum only ensures the mixture is balanced and substantially free from any traces of air bubbles. Since air bubbles do not combine well with curing the silicone, there can be cracks in the silicone after the curing if air is not removed.

In certain embodiments, a 50:50 mixture of silicone components A and B plus the balanced and thoroughly mixed oil and AZCH mixture is mixed again and again. When the stirring is applied, often air mixes in with it. Therefore, the vacuum applies to remove air; with no solvent evaporation.

When fabricating liners without an antiperspirant dispersed therein, usually, all components are put into a single mixture and then mixed together. But, since the AZCH is in powder form, it is first mixed with the oil to ensure a thorough mixture.

### Illustrative Example 5

Fabricating the liner may be carried out as follows. For each liner size, three mold sections comprising two outer mold sections and an inner mold section are provided. The first shot outer mold section is configured to give a thickness of the liner inner layer 18 while the second shot outer mold section is configured to provide a thickness of the outer layer 20 covering the inner layer 18, the matrix 28 and the connecting element 24. The inner mold section is a male form conforming to the inner surface of the liner. The outer mold sections are female molds that fit over the inner mold and provide a space between the inner and outer mold sections for receiving liquid silicone elastomer injected under pressure between the mold sections during the liner mold or casting process.

To form the inner layer 18, the inner mold is kept at a steady temperature slightly above ambient or room temperature (typically 35 C). The first shot outer mold is placed over the inner mold, centered and secured. Silicone material forming the inner layer is injected into the top of the outer mold section under a pressure of 40-100 psi and the mold is filled. The temperature is elevated to a desired level (70-125 C.) for curing or vulcanizing the first layer and then lowered after the silicone material has set.

The first shot outer mold section is then removed leaving the cured inner layer 18 of silicone elastomer exposed on the inner mold fixture. The correct size prosthesis connecting element 24 is placed on top of the inner layer (typically the inner mold element extends in an upright direction with the liner inverted) and a length of matrix material in a woven tubular stockinette is placed over the connecting element and stretched over the outer surface over a length of the inner layer of silicone material.

The second shot outer mold section is then placed over the inner mold section, the inner layer of silicone elastomer, the connecting element and the matrix material, and is centered and secured. A small centering screw may be tightened through the injection port for the silicone material of the outer second shot mold section which extends into the threaded socket 26 of the connecting element 24 to center and secure the connecting element 24 while the second shot mold is being filled. Silicone elastomer used to form the outer layer 20 of the liner is then injected between the outer surface of the inner layer of silicone and the outer second shot mold to fill the mold. The temperature is elevated until the desired curing or vulcanizing temperature (70-125 C) material has set. The outer mold section is then removed, leaving the cured, dual layered liner exposed on the inner mold section with the contained connecting element and matrix material in the liner.

The second layer of silicone material bonds intimately with the inner layer during the second molding procedure so the two layers are contiguous and joined virtually seamlessly over the outer surface of the inner layer, with the matrix 28 material filled with silicone elastomer material constituting the outer layer 20 of the liner. The connecting element 24 is intimately bonded between the two layers as well so the entire structure of the liner is integrally connected together.

The molded liner is then removed from the inner mold section and prepared for packing and shipment.

In forming the inner layer, parts A and B are mixed together before injection in the mold in a 1:1 ratio by weight. In at least one embodiment, an antiperspirant is mixed with a silicone-oil; a vacuum is applied on it. The antiperspirant-oil mixture is combined with the silicone components (A and B) and the vacuum is applied on it again.

When forming the outer layer 20, parts A and B of the silicone elastomer are mixed in a ratio of 1:1 to cause crosslinking by hydrosilylation, with the inhibitor allowing control of the rate of crosslinking of the two parts when mixed together. In at least one embodiment, an antiperspirant is mixed with a silicone-oil and a vacuum is applied on it. The antiperspirant-oil mixture is combined with the silicone components (A and B) and the vacuum is applied on it again.

One will appreciate, however, that the present disclosure is not so limited, and that other methods known in the art for dispensing, dispersing, or otherwise embedding compounds or substances within a carrier material are contemplated. Any such method may be used, according to the disclosure, to include an antiperspirant with and/or in a liner for use with a prosthetic or orthotic device.

Embodiments of the disclosure may be in other forms without departing from its spirit or essential characteristics. The described embodiments are to be only as illustrative and not restrictive. While exemplary embodiments have been described, persons skilled in the art may change structural, conceptual, instructional, and/or other details of the preferred or other embodiments without departing from the scope of the disclosure. The disclosure is, therefore, indicated by the appended claims rather than by the foregoing description. All changes that come within the meaning and range of equivalency of the claims are to be embraced within their scope.

We claim:

1. A method of manufacturing a liner for use with a prosthetic or orthotic device, comprising the steps of:
combining an antiperspirant agent with a silicone oil to form a first mixture;
mixing the first mixture under vacuum to form a balanced mixture, wherein air is substantially removed from the first mixture during mixing, and wherein the balanced mixture comprises a combination of the antiperspirant agent and the silicone oil and from which air has been substantially removed;
adding at least one silicone component to the balanced mixture to form a second mixture;
mixing the second mixture under vacuum to form a complete mixture, wherein air is substantially removed from the second mixture during mixing, and wherein the complete mixture comprises a combination of the balanced mixture and the at least one silicone component and from which air has been substantially removed; and
curing the complete mixture to form a silicone product such that the antiperspirant agent is dispersed in the silicone product.

2. The method of claim 1, wherein the antiperspirant agent is provided as a powder or anhydride.

3. The method of claim 1, wherein the antiperspirant agent comprises an aluminum salt.

4. The method of claim 3, wherein the aluminum salt comprises aluminum zirconium tetrachlorohydrex glycine.

5. The method of claim 1, wherein the silicone product comprises a liner for use with a prosthetic or orthotic device, and wherein the liner is capable of reducing perspiration of a body part to which the liner is attached by at least 20% compared to a liner that does not include an antiperspirant agent dispersed therein.

6. The method of claim 1, wherein the silicone product is configured for attachment to an amputated residual upper or lower limb.

7. A liner for use with a prosthetic or orthotic device, comprising:
an antiperspirant dispersed in carrier material, wherein:
the carrier material is configured for attachment to a body part;
the carrier material and antiperspirant are configured to permit the antiperspirant to leach from the carrier material and onto the body part to which the carrier material is attached; and
the antiperspirant is capable of reducing perspiration of the body part to which the carrier material is attached by at least 20% compared to a carrier material that does not include an antiperspirant dispersed therein.

8. The liner of claim 7, wherein the dispersion of the antiperspirant in the carrier material is substantially uniform.

9. The liner of claim 7, wherein the antiperspirant is configured to reduce perspiration by associating with moisture, entering a sweat gland duct cell, and causing said cell to swell, thereby limiting an amount of perspiration that can be secreted from a sweat gland.

10. The liner of claim 7, wherein the antiperspirant comprises a hygroscopic substance.

11. The liner of claim 7, wherein the antiperspirant comprises an aluminum-based compound.

12. The liner of claim 11, wherein the aluminum based compound comprises an aluminum salt.

13. The liner of claim 12, wherein the aluminum salt comprises aluminum zirconium tetrachlorohydrex glycine.

14. The liner of claim 7, wherein the carrier material is capable of elastic attachment to the body part.

15. The liner of claim 7, wherein the carrier material comprises a silicone-based compound.

16. The liner of claim 7, wherein the carrier material comprises a cured two-component silicone-based compound.

17. The liner of claim 7, wherein the carrier material and antiperspirant are configured to permit the antiperspirant to leach from the carrier material and onto the body part to which the carrier material is attached over an extended period of time.

18. The liner of claim 7, wherein the body part comprises an amputated residual upper or lower limb.

19. A liner for use with a prosthetic or orthotic device configured for being worn by a body part, comprising:
a liner body portion formed from an elastomeric material;
an antiperspirant incorporated and/or embedded in the elastomeric material of the liner body portion in a substantially uniform manner, and configured to permit the antiperspirant to leach from the liner body portion and onto the body part wearing the liner; and
wherein the antiperspirant is capable of reducing perspiration of the body part to which the carrier material is attached by at least 20% compared to the elastomeric material that does not include an antiperspirant dispersed therein.
wherein the antiperspirant is selected from the group consisting a hygroscopic substance, and an aluminum-based compound.

20. The liner of claim 19, wherein the carrier material comprises a silicone-based compound.
